(19) United States
(12) Patent Application Publication
(10) Pub. No.: US 2010/0112544 A1
(43) Pub. Date: May 6, 2010

(54) MODIFIED ROUTES OF LIPID METABOLISM, MEASURING OXIDIZED LDL AFTER FAT LOADING

(76) Inventor: Markku Ahotupa, Turku (FI)

Correspondence Address: BIRCH STEWART KOLASCH & BIRCH, PO BOX 747, FALLS CHURCH, VA, 22040-0747, US

(21) Appl. No.: 12/595,426

(22) PCT Filed: Apr. 9, 2008
(86) PCT No.: PCT/FI2008/050175

§ 371 Date: Oct. 9, 2009

(30) Foreign Application Priority Data
Apr. 11, 2007 (FI) 20075244

(51) Int. Cl. C12Q 1/00; G01N 33/02

(52) U.S. Cl. 435/4; 436/20

(57) ABSTRACT

The present invention relates to monitoring of lipid metabolism and is, particularly, directed to a test for estimating the individual susceptibility of a subject to the effect of oxidized dietary lipids. A high calorie and high fat meal were given to subjects and postprandial levels of oxidized low-density lipoproteins (LDL) were measured in the plasma of the subjects, indicating susceptibility to atherosclerotic events and insulin resistance. Food were also analysed for peroxide lipid content indicating LDL oxidizing potential of said foodstuff in a subject after consumption.

### FIELD OF THE INVENTION

The present invention relates to monitoring of lipid metabolism and is, particularly, directed to a test for estimating the individual susceptibility of a subject to the effect of oxidized dietary lipids.

### BACKGROUND OF THE INVENTION

The physiological function of lipoprotein particles is to transport lipids and lipid soluble material in the bloodstream to and from the liver. Low density lipoprotein (LDL) is the main transporter of cholesterol to the peripheral tissues, while excess tissue cholesterol is returned to the liver by reverse cholesterol transport mediated by high density lipoprotein (HDL). In parallel with the transport functions, high LDL cholesterol is associated with elevated risk of cardiovascular disease, while high HDL cholesterol appears to be protective.

Along with the native lipids, LDL particles are known to contain products of lipid peroxidation. It is now well established that oxidatively modified lipids in LDL are the main reason for atherogenicity of LDL¹. Oxidatively modified LDL contributes to atherogenic processes by multiple mechanisms. Oxidized LDL lipids are directly linked with macrophage accumulation, regulation of macrophage activity and foam cell formation in vessel wall², and oxidized lipids are strongly involved in activation of atherosclerosis-related gene groups³.

Each LDL particle contains approximately 1600 cholesteryl esters, 170 triglycerides, 700 phospholipids, 600 free cholesterol molecules, and one apolipoprotein B molecule. Lipids and lipid soluble material in LDL may be of endogenous or exogenous (food lipids) origin, while apolipoprotein B is synthesized in the liver. The lipid moieties of LDL are in continuous dynamic change between other lipoprotein particles, peripheral tissues and the liver, while the integral apolipoprotein B remains the same throughout the lifespan of the LDL particle. Measurement of “oxidized LDL” can be based on determination of oxidized LDL lipids or, alternatively, oxidatively modified apolipoprotein B. When the measurement is based on oxidized LDL lipids, it is indicative of the total lipid peroxide load being carried (as determined by the transport function of LDL) to peripheral tissues. On the contrary, quantification of oxidative modifications of apolipoprotein B is indicative of protein-modifying free radical activity in vessel wall. Thus, even though the term “oxidized LDL” can be applied to both ways of determining LDL oxidation, the biological mechanisms of which they are indicative, are not the same. Importantly, the potentially toxic and atherogenic lipid peroxidation products are those harmful molecular species which are responsible for the multiple pathophysiological effects of oxidized LDL.

It has been generally assumed that the oxidative modifications of LDL are due to endogenous free radical reactions, caused e.g. by metal ions, superoxide anion, nitric oxide, lipoxygenase and myeloperoxidase⁴. However, actual contribution of any of these mechanisms has not been verified in vivo. The present application deals with the role of an exogenous source of oxidized LDL lipids: lipid peroxidation products in food, which are readily found in edible fats and known to be formed during cooking⁵.

### SUMMARY OF THE INVENTION

In the present study we show that a meal rich in lipid peroxides can be the source of oxidized lipids and result in substantially increased postprandial concentration of oxidized LDL lipids.

Consequently, based on the data presented in this application a clinical fat load test can be developed for estimation of individual susceptibility of a subject to
1. direct atherogenic effect of oxidized dietary lipids,
2. adipogenic (obesity-increasing) effect of oxidized dietary lipids, and/or
3. insulin resistance due to oxidized dietary lipids.

The clinical fat load test comprises a standard high-fat meal, and postprandial analysis of oxidized LDL lipids. For the purposes of this invention, a standard high-fat meal is a meal containing about 900 kcal and about 50 g of fat. The lipid peroxide content of such a meal is over 1000 μmol, but it may be up to 1300 μmol. In specific, in the fat load test of the invention a standard high-fat meal is fed to a subject, and the postprandial concentration of oxidized LDL lipids is analysed in a biological sample obtained from said subject. The biological sample is appropriately a blood sample, preferably a serum or plasma sample.

Secondly, in addition to the estimation of individual susceptibility, the clinical fat load test can be applied to testing of drugs or dietary interventions for the prevention of the post-prandial increase of oxidized LDL lipids.

A third option is to develop a test for estimation of the LDL-modifying (oxidized LDL lipids-generating) potential of various meals and foodstuffs, and hence their potential for increasing atherogenicity, adipogenicity and insulin resistance-inducing capacity of LDL. The test for the lipid peroxide-dependent LDL-modifying potential of meals/foodstuffs comprises homogenization of the material, extraction of lipids, and analysis of oxidized food lipids by conventional methods for lipid peroxides, such as spectrophotometric determination of conjugated dienes. In the test a high lipid peroxide content of the meal or foodstuff material is indicative of a high lipid peroxide-dependent LDL-modifying potential of the meal or foodstuff.

### BRIEF DESCRIPTION OF DRAWINGS

FIG. 1. Postprandial concentration of oxidized LDL lipids after a high-fat meal.

FIG. 2. Postprandial oxidized LDL lipids/LDL cholesterol ratio after a high-fat meal.

FIG. 3A. Total lipid peroxide contents of a hamburger meal and an oatmeal. Lipid per-oxides were analysed by the spectrophotometric diene conjugation method in lipid extracts of the meals.

FIG. 3B. Postprandial levels of circulating oxidized LDL lipids after consumption of the hamburger meal (N=19) or the oatmeal (N=16).

### DETAILED DESCRIPTION OF THE INVENTION

### Methods

The aim of the study was to investigate whether oxidized dietary lipids, abundantly present in high-fat meals, are able to incorporate into LDL particles in human volunteers, and thus directly contribute to atherogenicity of LDL. An ordinary hamburger meal was used as the test meal.

Subjects In trial 1 (hamburger meal) the subjects were healthy men aged between 30 and 60 years. Subjects provided an informed consent prior entering the study. A total of 19 subjects participated in the study. In trial 2 (a comparative study with oatmeal porridge), 16 men were recruited using the same criteria.

Test diets Before the study the subjects were asked to maintain their normal lifestyle habits and not to make any changes to the diet or in the physical activity. The test subjects had a standardized breakfast in the morning at 8 o'clock. The subjects were not allowed to eat anything until 11 o'clock when the baseline (0 h) blood sample was drawn. After the baseline blood sample subjects consumed a standard hamburger meal and 4 dl of fruit juice. Further blood samples were taken at 2, 4 and 6 h time points after baseline measurement. The subjects were not allowed to eat, but they were allowed to drink water during the 6 h test period. The test meal in trial 2 was ordinary oatmeal porridge. In this study blood samples were taken before (0 h) and 1, 2 and 4 h after the meal. Choosing of the time points was based on assumption of faster absorption than in case of the hamburger meal.

### Determination of Oxidized LDL Lipids, and Other Analytical Procedures

Analysis of oxidized LDL lipids was based on determination of the baseline level of conjugated dienes in LDL lipids. The assay method consists of precipitation of LDL, extraction of LDL lipids, and spectrophotometric analysis of conjugated dienes in LDL lipids at 234 nm. Serum total (free and protein-bound) malondialdehyde was determined as the 2,4-dinitrophenylhydrazine derivative by HPLC with 1,1,3,3-tetraethoxypropane as the standard. Total peroxyl radical trapping antioxidant potential was estimated ex vivo by the potency of serum samples to resist ABAP-induced peroxidation. Trolox served as a standard radical scavenger. Paraoxonase activity was determined using paraoxon (O,O-diethyl-O-p-nitrophenylphosphate) as the substrate and measuring the absorbance at 412 nm. Serum insulin was measured with time-resolved fluoroimmunoassay method. Determination of serum glutathione was based on the method of Seville. Antioxidant vitamin (α-tocopherol, γ-tocopherol, β-carotene, retinol, ubiquinol-10) concentrations were analysed by standard HPLC procedures with UV-detection.

### Determination of Oxidized Food Lipids

The meals were weighed and, subsequently, they were minced and homogenized. Lipids were extracted with chloroform-methanol. The extract was evaporated to dryness and redissolved in cyclohexane. Measurement of the lipid peroxide content was based on spectrophotometric determination of diene conjugation at 234 nm. Absorbance units were converted to molar units using the molar extinction coefficient 2.95×10⁴ M⁻¹cm⁻¹.

### Results and Discussion

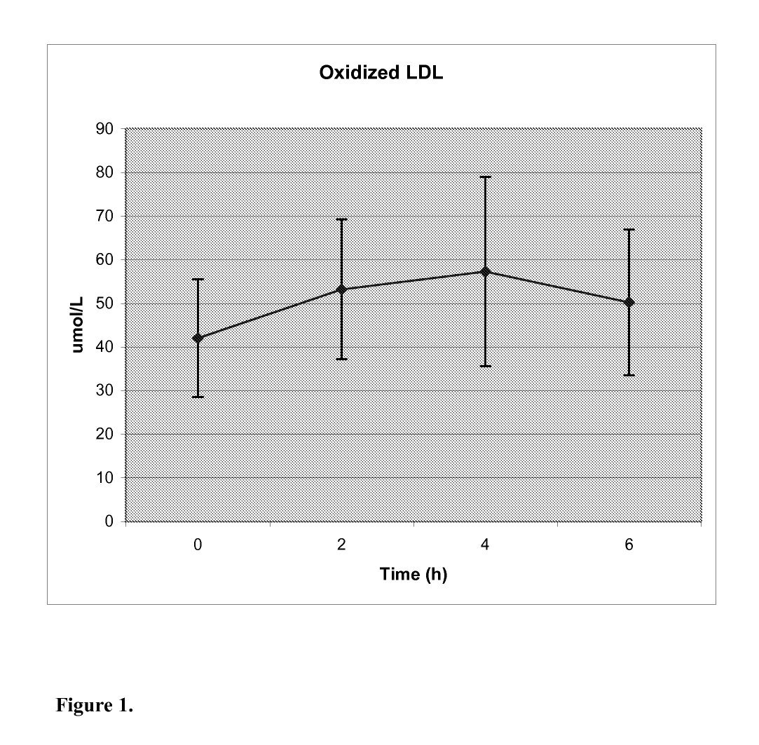

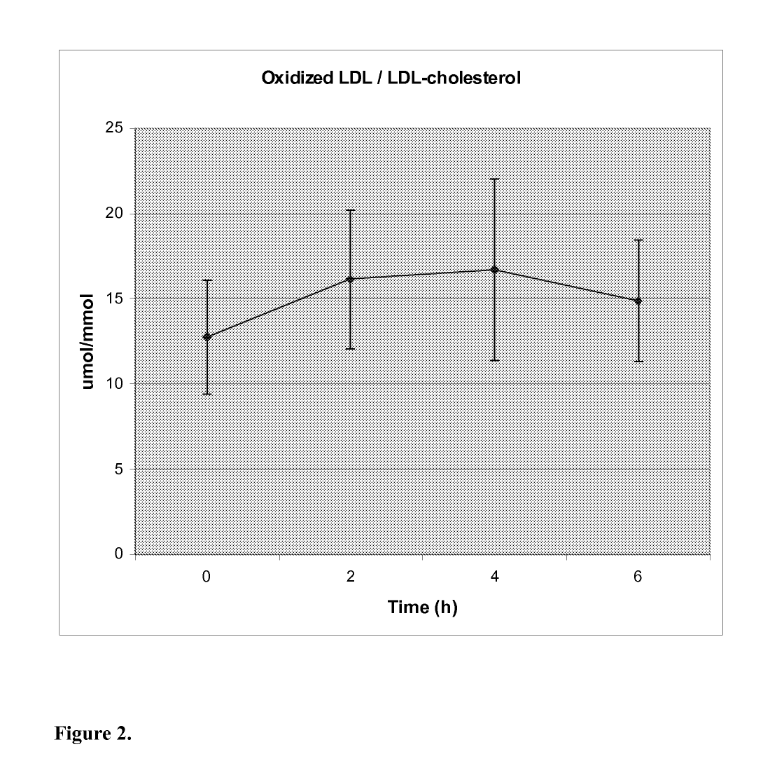

The various markers of LDL oxidation, oxidative stress and antioxidant functions were determined before the hamburger meal and monitored for 6 h postprandially. The concentration of oxidized LDL lipids (FIG. 1), and the ratio of oxidized LDL lipids:LDL cholesterol (FIG. 2) were markedly elevated throughout the postprandial period; peak values (36% and 30% increases, respectively) were detected 4 h after the test meal. This finding thus shows that fatty meals rich in lipid peroxides play an important role in generating and upholding oxidative modifications in atherogenic lipoproteins. This is highly significant with regard to the risk of atherosclerosis, since oxidized lipids in circulating LDL are known to be surprisingly strongly associated with coronary⁶, carotid^{7,8} and brachial⁷ atherosclerosis, hypertension⁷ and arterial functions^{8,9}. Furthermore, in line with the beneficial effects of risk management programs, serum concentrations of oxidized LDL lipids can be reduced by weight reduction^{10,11}, physical activity^{12,13}, statin treatment¹⁴⁻¹⁶ and dietary interventions^{14,17}. It is common to all these studies that the oxidized LDL lipids are more sensitive and specific indicators of the risk than the customary lipid markers of atherosclerosis. In comparison with previous studies on risk management programs, the magnitude of the hamburger meal-induced increase in oxidized LDL lipids was significant, comparable to that caused by 10 kg overweight¹⁰. This means that oxidized dietary lipids may substantially affect the risk of atherosclerosis.

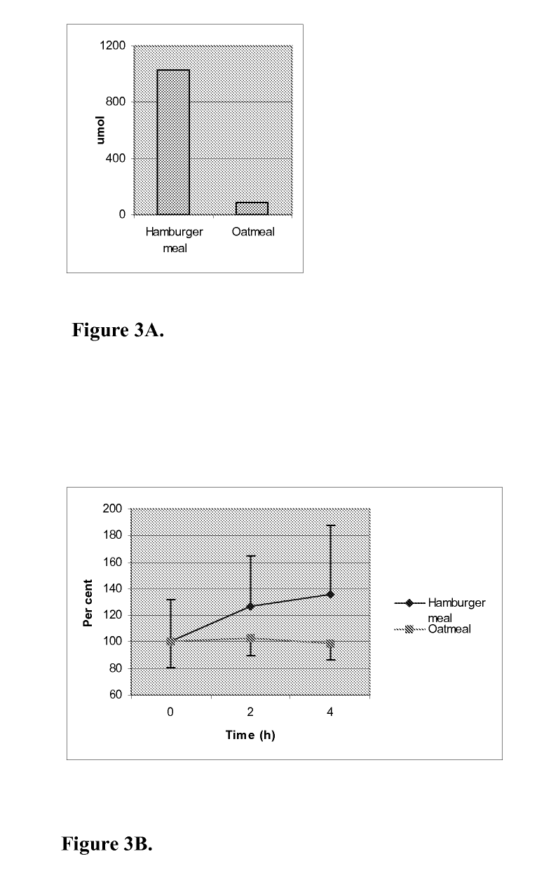

In support for the dietary origin of oxidized LDL lipids, the postprandial rise in oxidized LDL lipids was found to be related to the lipid peroxide content of the meal. The lipid peroxide content of hamburger and oat meals were 1030 μmol and 80 μmol, respectively (FIG. 3A), and while the hamburger meal elevated the concentration of oxidized LDL lipids by 36%, oat meal had no effect (FIG. 3B). These findings also mean that the LDL oxidation increasing effect of foodstuffs can be estimated by analysing their lipid peroxide content.

To investigate whether the observed oxidative changes were due to alterations in oxidative stress or antioxidant defense, indicators of these were also analysed. Blood samples were analysed for serum malondialdehyde, total (peroxyl radical scavenging) antioxidant potential, paraoxonase activity, antioxidant vitamin and glutathione concentrations. The effects of the hamburger meal on serum antioxidant functions were modest and do not explain the LDL modifications or oxidative stress.

The postprandial rise in oxidized LDL lipids was strongly correlated with elevations of serum insulin concentrations (Table 1). The association with insulin is in line with a previous finding showing that the amount of peroxides present in LDL particles is significantly correlated with insulin resistance¹⁸. This finding means that individuals with high post-prandial oxidized LDL lipid responses are more susceptible to insulin resistance.

There were large interindividual variations among test subjects in their responses to the hamburger meal. The range of the area under curve (0-6 h) for oxidized LDL lipids (oxLDLauc) was 21.5-87.5 μmol·L⁻¹. The oxLDLauc was strongly associated with BMI (Table 1), which is highly interesting in light of a recent experimental (in vitro) finding showing that exposure of adipocytes to oxidized LDL lipids results in a high proliferation rate, low level of apoptosis and impaired differentiation¹⁹. The finding of the present study thus implies that individuals with high oxLDLauc response are more prone to adipogenic effects of oxidized dietary lipids than those with a lower response.

TABLE 1
Correlation of postprandial oxidized LDL lipid concentrations
(area under the curve 6 h) with insulin concentration (area
under the curve 6 h) and BMI of test subjects in trial 1.

| | | |
|---|---|---|
| Insulin | r = 0.526 | p = 0.010 |
| BMI | r = 0.637 | p = 0.001 |

### REFERENCES

1. Stocker, R. & Keaney, J. F. Jr. Role of oxidative modifications in atherosclerosis. Physiol Rev 84, 1381-1478 (2004).
2. Birukov, K. G. Oxidized lipids: the two faces of vascular inflammation. Curr Atheroscler Rep 8, 223-231 (2006).
3. Furnkranz, A. et al. Oxidized phospholipids trigger atherogenic inflammation in murine arteries. Arterioscler Thromb Vasc Biol 25, 633-638 (2005).
4. Berliner, J. A. & Heinecke, J. W. The role of oxidized lipoproteins in atherogenesis. Free Radic Biol Med 20, 707-727 (1996).
5. Kubow, S. Routes of formation and toxic consequences of lipid oxidation products in foods. Free Radic Biol Med 12, 63-81 (1992).
6. Vasankari, T. et al. Oxidized LDL and thickness of carotid intima-media are associated with coronary atherosclerosis in middle-aged men: lower levels of oxidized LDL with statin therapy. Atherosclerosis 155, 403-412 (2001).
7. Toikka, J. O. et al. Increased arterial intima-media thickness and in vivo LDL oxidation in young men with borderline hypertension. Hypertension 36, 929-933 (2000).
8. Raitakari, O. T. et al. Reduced myocardial flow reserve relates to increased carotid intima-media thickness in healthy young men. Atherosclerosis 156, 469-475 (2001).
9. Toikka, J. et al. Large artery elastic properties in young men: relationships to serum lipoproteins and oxidized low-density lipoprotein cholesterol. Arterioscler Thromb Vasc Biol 19, 436-441 (1999).
10. Vasankari, T. et al. Reduced LDL oxidation after weight reduction in obese premenopausal women. Int J Obes Relat Metab Disord 25, 205-211 (2001).
11. Raitakari, M. et al. Weight reduction with very-low caloric diet and endothelial function in overweight adults. Role of plasma glucose. Arterioscler Thromb Vasc Biol 24, 124-128 (2004).
12. Vasankari, T. J., Kujala, U., Vasankari, T. M. & Ahotupa, M. Reduced oxidized LDL levels after a ten-month exercise program. Med Sci Sports Exerc 30, 1496-1501 (1998).
13. Vuorimaa, T., Ahotupa, M., Irjala, K. & Vasankari, T. Prolonged, acute exercise reduces moderately oxidized LDL in endurance trained men. Int J Sports Med 26, 420-425 (2005).
14. Jula, A. et al. Effects of diet and simvastatin on serum lipids, insulin, and antioxidants in hypercholesterolemic men. JAMA 287, 598-605 (2002).
15. Janatuinen, T. et al. The effect of pravastatin treatment on LDL oxidation and myocardial blood flow in young adults with type 1 diabetes. Arterioscler Thromb Vasc Biol 24, 1303-1308 (2004).
16. Vasankari, T. et al. Effects of statin therapy on circulating conjugated dienes, a measure of LDL oxidation. Atherosclerosis 179, 207-209 (2005).
17. Jenkins, D. J. A. et al. Dose response of almonds on coronary heart disease risk factors: blood lipids, oxidized low-density lipoproteins, lipoprotein(a), homocysteine and pulmonary nitric oxide. A randomized, controlled, crossover trial. Circulation 106, 1327-1332 (2002).
18. Carantoni, M. et al. Relationship between insulin resistance and partially oxidized LDL particles in healthy, nondiabetic volunteers. Arterioscler Thromb Vasc Biol 18, 762-767 (1998).
19. Masella, R. et al. Oxidized LDL modulate adipogenesis in 3T3-L1 preadipocytes by affecting the balance between cell proliferation and differentiation. FEBS Letters 580, 2421-2429 (2006).

1-6. (canceled)

7. A clinical fat load test, comprising the steps of
feeding a standard high-fat meal to a subject, and
analysing the postprandial concentration of oxidized low density lipoprotein (LDL) lipids in a biological sample obtained from the subject.

8. A clinical fat load test, comprising the steps of
(a) analysing the concentration of oxidized low density lipoprotein (LDL) lipids in a biological sample obtained from a subject, in order to determine a baseline level value of oxidized LDL lipids,
(b) feeding a standard high-fat meal to the subject,
(c) analysing the postprandial concentration of oxidized LDL lipids in a biological sample obtained from the subject after the high-fat meal, in order to determine a postprandial value of oxidized LDL lipids, and
(d) comparing the values obtained in steps (a) and (c),
wherein postprandial increase of oxidized LDL lipids indicates that oxidized dietary lipids are incorporated into LDL particles.

9. The clinical fat load test according to claim 7 or 8, wherein the standard high-fat meal consists of about 900 kcal and about 50 g of fat.

10. The clinical fat load test according to claim 7 or 8, wherein the biological sample is a serum or plasma sample.

11. The clinical fat load test according to claim 7 or 8 for estimating individual susceptibility of the subject to direct atherogenic effect of oxidized dietary lipids.

12. The clinical fat load test according to claim 7 or 8 for testing of drugs or dietary interventions for prevention of the postprandial increase of oxidized LDL lipids.

13. A test for estimation of the lipid peroxide-dependent LDL-modifying potential of meals and foodstuffs, comprising
homogenizing the meal or foodstuff material,
extracting lipids from the material, and
analysing the lipid peroxide content of the lipids as extracted,
wherein a high lipid peroxide content of the meal or foodstuff material is indicative of a high lipid peroxide-dependent LDL-modifying potential of the meal or foodstuff.
